(19) United States
(12) Patent Application Publication
(10) Pub. No.: US 2016/0151520 A1
(43) Pub. Date: Jun. 2, 2016

(54) DEVICE FOR STERILIZING EVAPORATOR CORE OF AIR CONDITIONING SYSTEM

(71) Applicants: HYUNDAI MOTOR COMPANY, Seoul (KR); HALLA VISTEON CLIMATE CONTROL CORP., Daejeon (KR)

(72) Inventors: Sang Shin LEE, Suwon-si (KR); Myung Hoe KIM, Seoul (KR); Yong Jun JI, Daejeon (KR); Jae Ho KIM, Daejeon (KR)

(21) Appl. No.: 14/862,112

(22) Filed: Sep. 22, 2015

(30) Foreign Application Priority Data
Dec. 2, 2014 (KR) 10-2014-0170195

(51) Int. Cl. A61L 2/08

(52) U.S. Cl. CPC A61L 2/088

(57) ABSTRACT

A device for sterilizing an evaporator core of an air conditioning system includes a sterilizer is mounted at a front end of the eva core for sterilization and deodorization of the eva core. The sterilizer is mounted at one side of a wall surface of an air channel formed between a blower which blows air toward the eva core and the eva core to prevent an air volume of an air channel from being reduced.

### CROSS-REFERENCE TO RELATED APPLICATION

This application claims under 35 U.S.C. §119(a) the benefit of priority to Korean Patent Application No. 10-2014-0170195 filed on Dec. 2, 2014, the entire content of which is incorporated herein by reference.

### TECHNICAL FIELD

The present disclosure relates to a device for sterilizing an evaporator core of an air conditioning system. More particularly, the present disclosure relates to a device for sterilizing an evaporator core capable of removing germs and mold from the evaporator core which are main causes of smell in an air conditioning system and performing a deodorization function using a photocatalytic reaction.

### BACKGROUND

A vehicle is equipped with an air conditioning system called a heating ventilating air conditioning (HVAC) to make an inner space of the vehicle comfortable.

The air conditioning system includes an evaporator core (hereinafter, referred to as “eva core”) which cools air supplied from a ventilating unit using a refrigerant, a heater core which heats air using cooling water of an engine, and a blower unit which supplies cold air and hot air generated by the eva core and the heater core to an interior of the vehicle.

The air conditioning system can selectively pass air introduced into a vehicle through the eva core and the heater core to generate cold air or hot air and supplies the cold air or the hot air to the interior of the vehicle by the blower unit to cool and heat the interior of the vehicle.

In the eva core, the refrigerant in a compressed liquid state is vaporized by passing through an expansion valve in front of a high pressure pipeline, and the heat generated at the time of the vaporization may prevent heated air from being introduced into the vehicle but instead generate cold air.

Further, condensed water is generated due to a vaporization phenomenon of the refrigerant. Here, the condensed water is condensed and vaporized at the time of turning on and off a cooler, and thus, hydrophilic particles and/or hydrophobic particles are evaporated, thereby causing an unpleasant smell.

Mold, germs, bacteria, and the like are propagated in the eva core due to moisture and thus are introduced into the interior of the vehicle, along with the unpleasant smell.

The above information disclosed in this Background section is only for enhancement of understanding of the background of the disclosure, and therefore, it may contain information that does not form the prior art that is already known in this country to a person of ordinary skill in the art.

### SUMMARY OF THE DISCLOSURE

The present disclosure has been made in an effort to solve the above-described problems associated with prior art. An aspect of the present inventive concept provides a device for sterilizing an evaporator core of an air conditioning system capable of implementing an ultraviolet ray sterilization function and a deodorization function of the eva core using an UV light source having the ultraviolet ray sterilization function and a photocatalytic unit having a light transmitting function.

According to an exemplary embodiment of the present inventive concept, a device for sterilizing an evaporator (eva) core of an air conditioning system includes a sterilizer for the eva core mounted at a front end of the eva core for sterilization and deodorization of the eva core. The sterilizer is mounted at one side of a wall surface of an air channel formed between a blower which blows air toward the eva core and the eva core to prevent an air volume of the air channel from being reduced.

The sterilizer may be mounted at an incline which is a slope from the front end of the eva core toward the eva core when the sterilizing means is installed at one side of the wall surface of the air channel to illuminate an ultraviolet light emitted from the sterilizer onto the entire surface of the eva core.

The sterilizer may include a photocatalytic unit having light transmittance and an UV light source which irradiates the ultraviolet light to the photocatalytic unit.

The sterilizer may illuminate the ultraviolet light transmitting the photocatalytic unit to the entire surface of the eva core and supply radical ions generated by a photocatalytic reaction generated in the photocatalytic unit by the ultraviolet light to the entire surface of the eva core by ventilation of a blower.

The UV light source may be an ultraviolet-C light-emitting diode (UV-C LED) having a sterilization function.

A rear side of the UV light source may have a heat radiator for radiating heat from the UV light source to outside.

According to the device for sterilizing an evaporator core of an air conditioning system of the present disclosure, the ultraviolet light emitted from the UV light source transmits the photocatalytic unit to be illuminated to the entire surface of the eva core to implement the deodorization function using the photocatalytic unit and the sterilization function using the UV light source. The device for sterilizing the eva core is mounted on the wall surface of the air channel, and thus, resistance of the air channel due to the device for sterilizing the eva core is removed to prevent the air volume of the air channel from being reduced.

Other aspects and embodiments of the inventive concept are discussed infra.

### BRIEF DESCRIPTION OF THE DRAWINGS

The above and other features of the present disclosure will now be described in detail with reference to certain exemplary embodiments thereof illustrated in the accompanying drawings which are given hereinbelow by way of illustration only, and thus are not limitative of the present disclosure.

FIG. 1 is a schematic diagram illustrating a mounting position of a sterilizer for an evaporator core according to an exemplary embodiment of the present inventive concept in an air conditioning system.

FIG. 2 is an exploded perspective view illustrating the sterilizer according to the exemplary embodiment of the present inventive concept.

FIG. 3 is a cross-sectional view illustrating the sterilizer according to the exemplary embodiment of the present inventive concept.

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes will be determined in part by the particular intended application and use environment.

In the figures, reference numbers refer to the same or equivalent parts of the present disclosure throughout the several figures of the drawing.

### DETAILED DESCRIPTION

Hereinafter reference will now be made in detail to various embodiments of the present inventive concept, examples of which are illustrated in the accompanying drawings and described below. While the disclosure will be described in conjunction with exemplary embodiments, it will be understood that present description is not intended to limit the invention to those exemplary embodiments. On the contrary, the disclosure is intended to cover not only the exemplary embodiments, but also various alternatives, modifications, equivalents and other embodiments, which may be included within the spirit and scope of the invention as defined by the appended claims.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

Hereinafter, exemplary embodiments of the present inventive concept so as to be easily practiced by a person of ordinary skill in the art to which the present invention pertains will be described with reference to the accompanying drawings.

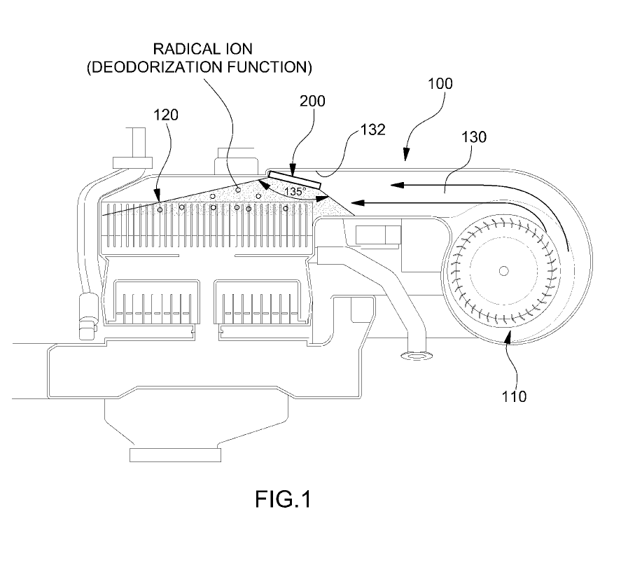

A sterilizer 200 for an evaporator (eva) core according to an exemplary embodiment of the present inventive concept is to effectively remove pollutants (germ, mold, etc.) which are a cause of smell of an air conditioning system. As illustrated in FIG. 1, the sterilizer 200 is installed in an air channel 130 which is formed between a blower 110 and an eva core 120 within a heating ventilating air conditioning (HVAC) system 100.

Air ventilated from the blower 110 (driven by a blower motor) to the eva core 120 flows in the air channel 130. Therefore, to prevent the air in the air channel 130 from being lost or the air channel 130 from being shut off, that is, to prevent an air volume supplied to the eva core 120 through the air channel 130 from being reduced or lost, the sterilizer 200 is attached to one side of a wall surface 132 (wall surface portion surrounding the air channel) of the air channel 130.

The sterilizer 200 for an eva core is mounted at the one side of the wall surface 132 surrounding the air channel 130 to remove (minimize) air resistance due to the sterilizer 200, thereby preventing flow impediment of the air ventilated to the eva core 120.

The sterilizer 200 for an eva core includes a UV light source 220 for sterilizing the eva core 120 and a photocatalytic unit 230 for deodorizing the eva core 120. The sterilizer 200 is inclined at a predetermined slope from a front end of the eva core 120 toward a rear end the eva core 120 when the sterilizer 200 is mounted at the one side of the wall surface 132 of the air channel 130 so as to irradiate ultraviolet light, which is irradiated from the UV light source 220, to the entire surface of the eva core 120.

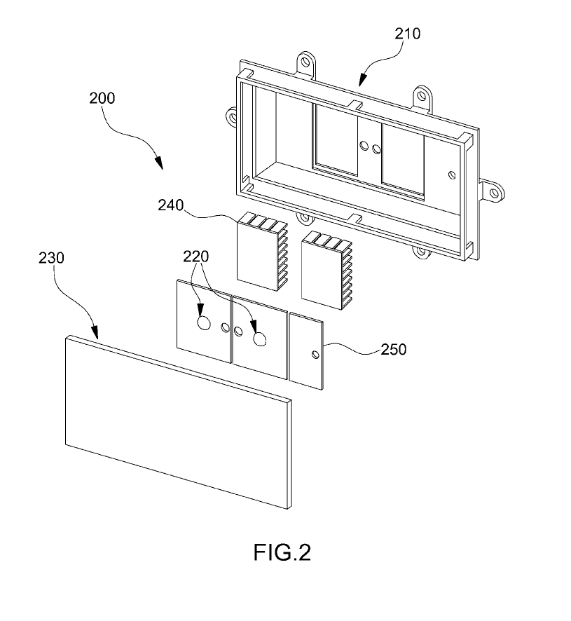

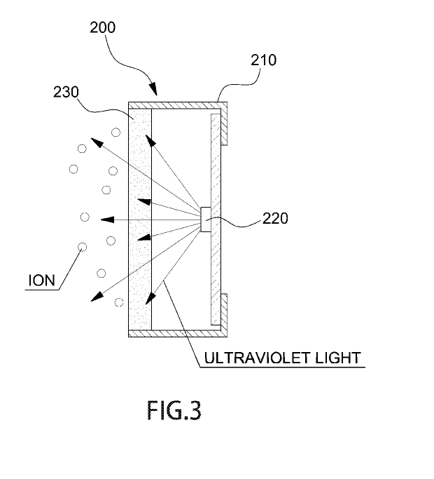

Referring to FIGS. 2 and 3, the sterilizer 200 for an eva core includes a main body 210 which is attached to the one side of the wall surface 132 of the air channel 130. The photocatalytic unit 230 is configured to transmit light and mounted at a front end of the main body 210. The UV light source 220 is installed at a rear end of the main body 210 at a predetermined interval from the photocatalytic unit 230 to irradiate the UV light to the photocatalytic unit 230.

Here, as the UV light source 220, an ultraviolet-C light-emitting diode (UV-C LED) having a sterilization function, and the like is used.

The UV-C LED has an excellent sterilization function to maximize a sterilization effect at the time of directly irradiating the UV light and has strong sterilizing ability to implement the sterilization function of microorganism in the air. The LED has a wide irradiation angle (approximately 130 to 135°) and thus irradiates ultraviolet light to the entire surface of the eva core at a predetermined distance (approximately 80 mm), thereby sterilizing smell substances (such as pollutants which are a cause of smell). Further, the LED has good durability and thus may be easily applied to the air conditioning system of a vehicle.

For example, as a result of testing and evaluation a sterilizing force of the UV-C LED, UV light is irradiated at microorganism for a predetermined time to confirm a removal rate of 100%.

The photocatalytic unit 230 generates ions (OH radicals) by the photocatalytic reaction which is generated by the ultraviolet light irradiated from the UV light source 220 and makes the generated ions contact a surface of the eva core 120 along with the air ventilated from the blower unit, thereby deodorizing the smell of the eva core 120.

The above eva core sterilization device may implement the sterilization function by the ultraviolet light from the UV light source 220 along with the deodorization function through radical ions generated by the photocatalytic reaction in the photocatalytic unit 230 by making the UV light emitted from the UV light source 220 transmit the photocatalytic unit 230 and be irradiated onto the entire surface of the eva core 120. In this case, the ultraviolet light transmitting the photocatalytic unit 230 is directly irradiated to the entire surface of the eva core 120 without being dispersed and lost.

The main body 210 of the sterilizater 200 has a rear portion provided with the UV light source 220 of which the rear portion has a heat radiator 240 such as a heat sink which absorbs heat from the UV light source 220 and radiates the absorbed heat to outside.

An LED drive 250 for safely driving the UV light source when it is supplied with power is connected to one side of the UV light source 220.

Although the embodiments of the present inventive concept are described above in detail, the scope of the present disclosure is not limited thereto. Therefore, various changes and improved forms by those skilled in the art using basic concepts of the present disclosure defined in the following claims belong to the protection scope of the present disclosure.

The inventive concept has been described in detail with reference to exemplary embodiments thereof. However, it will be appreciated by those skilled in the art that changes may be made in these embodiments without departing from the principles and spirit of the disclosure, the scope of which is defined in the appended claims and their equivalents.

What is claimed is:

1. A device for sterilizing an evaporator (eva) core of an air conditioning system, the device comprising:
a sterilizer for the eva core mounted at a front end of the eva core for sterilization and deodorization of the eva core,
wherein the sterilizer is mounted at one side of a wall surface of an air channel formed between a blower which blows air toward the eva core and the eva core to prevent an air volume of the air channel from being reduced due to the sterilizer.

2. The device of claim 1, wherein the sterilizer is mounted at an incline, which is a slope from the front end of the eva core toward a rear end of the eva core when the sterilizer is mounted at the one side of the wall surface of the air channel, to irradiate an ultraviolet light emitted from the sterilizer onto the entire surface of the eva core.

3. The device of claim 1, wherein the sterilizer includes a photocatalytic unit which transmits light and a ultraviolet (UV) light source which irradiates an ultraviolet light to the photocatalytic unit, and
the sterilizer irradiates the ultraviolet light transmitting the photocatalytic unit to the entire surface of the eva core and supplies radical ions, which are generated in the photocatalytic unit by the ultraviolet light, to the entire surface of the eva core by ventilation of the blower.

4. The device of claim 3, wherein the UV light source has a heat radiator at a rear side thereof, the heat radiator radiating heat from the UV light source to outside.

5. The device of claim 3, wherein the UV light source is an ultraviolet C light-emitting diode (UV-C LED) having a sterilization function.

6. The device of claim 3, wherein the sterilizer further includes a main body attached to the one side of the wall surface,
wherein the photocatalytic unit is mounted at a front end of the main body and the UV light source 220 is mounted at a rear end of the main body.

7. The device of claim 3, wherein the UV light source includes an LED drive at one side thereof for safely driving the UV light source.
